(19) United States
(12) Patent Application Publication
(10) Pub. No.: US 2004/0132177 A1
(43) Pub. Date: Jul. 8, 2004

(54) Thermoformed container for cell culture

(76) Inventor: Antoine Heron, Halluin (FR)

Correspondence Address: Paul R. Morico, Baker Botts L.L.P., 910 Louisiana Street, Houston, TX, 77002-4995, US

(21) Appl. No.: 10/742,658

(22) Filed: Dec. 19, 2003

(30) Foreign Application Priority Data
Dec. 20, 2002 (FR) FR 02/16439

(51) Int. Cl. C12M 1/12

(52) U.S. Cl. 435/297.5; 435/304.1; 435/305.4

(57) ABSTRACT

The invention relates to a container intended for use in cell culture. The container includes a first and a second sheet that are secured to one another in the vicinity of their periphery so as to form an interior volume intended to receive cells and at least one access route designed to allow the introduction and/or the recovery of cells. Each of the sheets includes at least one layer made of a polymer material that allows the cells to adhere. Moreover, at least one of the two sheets is thermoformed. The invention also relates to a system including at least two elements connected together as a closed circuit by a tube. One of the elements is a container of the invention. The invention also includes the use of such containers and systems for culturing adherent cells and/or cells that are suspended in the medium.

### PRIORITY CLAIM

The present application claims priority under 35 U.S.C. §119(d) to French Patent Application Ser. No. 02/16439, filed Dec. 20, 2002.

### FIELD OF THE INVENTION

The invention relates to a container intended for the culturing of cells, to a system having at least two elements connected together as a closed circuit by a tube, one of said elements being such a container, and also to the use of such a container or of such a system for culturing cells.

The container according to the invention is more particularly intended for the culturing of cells by adhesion to the inner surface of the container. The container may also be used for culturing cells in suspension in a medium contained in the container.

### BACKGROUND

The advent of in vitro cultures of cells that can be directly transplanted into humans required the development of different types of container for packaging said cultures. When used in a medical context (e.g. to prepare products for use in cell and gene therapy), these containers should confine the cells to reduce any risks of contamination and technical handling errors. The containers must therefore comply with strict good practice regulations for transfusable products with respect to closed packaging and cell transfer.

The use of flexible pouches for culturing cells used in human therapy has grown, particularly in the context of developing clinical protocols for the ex vivo expansion of hematopoietic cells derived from a sample of bone marrow, peripheral blood or umbilical cord blood. Flexible pouches comply with the required good practice regulations but have disadvantages with respect to culturing cells.

First, the flexibility of these pouches does not allow them to be conveniently stacked within an incubator.

Second, the flexibility of a pouch defines a culture surface that is flexible and therefore deformable as a function of the level of filling and the handling operations. This deformability causes zones of sedimentation and a heterogeneous distribution of the cells over the available culture surface.

Furthermore, the available culture surface is limited by the size of the pouch. In order to satisfy certain applications that require large culture surfaces, the increase in the size of a pouch or the multiplicity of small pouches considerably increases the difficulties encountered during handling.

Finally, the gas-permeable materials conventionally used for flexible pouches for cell culturing, generally polyethylene, polypropylene, fluoropolymer, and ethylene-vinyl acetate (EVA), only allow the culture of cells in suspension in the medium. These polymers do not allow the culturing of adherent cells, which considerably limits the possible applications because most cells of interest are cells that are cultured by adhesion.

In an attempt to overcome this problem, U.S. Pat. No. 6,297,046 discloses a flexible pouch intended for the culturing of cells, in particular by adhesion. Essentially, the pouch is formed by the association of two sheets that are themselves made of a complex of two films, one of which defines an adhesive inner surface for the cells. The lower flexibility of adhesive polymer films necessitates the use of very thin films to produce a flexible pouch, hence the need to complex them. However, the use of complex sheets limits the transparency of the pouch and therefore the possibility of observing cell development under a microscope. Besides the difficulties in producing the pouch described in said document, this pouch only aims to solve the problem of adhesion of the cells, whereas the user would prefer a global response to all of the problems mentioned above.

### SUMMARY OF THE INVENTION

The invention in particular aims to solve all of the drawbacks mentioned above by providing a container that complies with the good practice regulations mentioned above while allowing the culturing of cells by adhesion, by allowing the container to be stacked, and by allowing increased and homogeneous culture surfaces in a container of small size.

For this purpose, and according to a first aspect, the invention relates to a container intended for the culturing of cells including a first and a second gas-permeable sheet that are secured to one another in the vicinity of their periphery so as to form an interior volume intended to receive the cells. The container also includes at least one access route designed to allow the introduction and/or the recovery of the cells.

Each of the gas permeable sheets include at least one layer made of a polymer material that allows cells to adhere, and at least one of the two sheets is thermoformed.

When the container is formed from at least one thermoformed sheet of adhesive polymer material, it has several beneficial characteristics:

the container may have a defined exterior geometry, which allows a number of containers to be conveniently stacked within an incubator.

the container has a certain rigidity, which makes it possible to avoid creating zones of preferential sedimentation. This is makes it easier to obtain a homogenous distribution of the cells over the available culture surface.

In one particular embodiment, each sheet may be formed essentially of a polymer material that allows the cells to adhere. Because thermoforming is used, it is not necessary to use complex sheets.

According to one variant of the invention, at least one of the thermoformed sheets has ridges that are arranged in the interior volume of the container. Thermoforming makes it possible to produce such ridges arranged in the interior volume of the container. Thus, it is possible to considerably increase the available culture surface without increasing the size of the container and without increasing the volume of culture medium consumed. Ridges may form repeating or irregular, continuous or separate motifs.

According to one possible embodiment, the sheets may be secured to one another by welding in the vicinity of their periphery. For example, at least a first sheet may be thermoformed so as to have, in transverse section, the overall shape of a rectangle with rounded comers comprising a substantially flat base, a side wall and a peripheral wall that forms a rim.

The second sheet, which forms the upper wall of the container, may be:

either secured to the rim of the first sheet so as to be substantially flat;

or thermoformed so as to have a geometry which is the same as that of the first sheet, the sheets being secured facing one another by their rims.

The container may comprise at least one access route that communicates with the interior volume of the container through a wall of a thermoformed sheet, for example a side wall, peripheral wall or upper wall.

According to a second aspect, the invention relates to a system including at least two elements connected together as a closed circuit by a tube. At least one of said elements may be a container as described above. The tube may be connected at a first end of an access route of the container and at a second end to another element of the system, so as to allow the cells and/or fluids to pass between the elements of the system.

Finally, according to a third aspect, the invention relates to the use of a container or a system as described above for culturing adherent cells or cells that are in suspension in the medium.

### BRIEF DESCRIPTION OF THE DRAWINGS

The other characteristics of the invention emerge from the following description of specific embodiments with reference to the appended figures.

FIG. 1 is a schematic representation of a transverse section of a container formed by a thermoformed sheet and another sheet, the container being provided with two access routes.

FIGS. 2A, 2B and 2C are schematic representations of transverse sections of containers formed by a thermoformed sheet having ridges on its inner face and by another sheet, the containers being provided with one or two access routes.

FIGS. 3A-F are schematic representations of transverse sections of containers formed by two thermoformed sheets, which do or do not have ridges, the containers being provided with one or two access routes.

FIG. 4A is a schematic representation of a transverse section of a container formed by a thermoformed sheet and another sheet, the container being provided, on the peripheral wall of the thermoformed sheet, with one access route that is oriented vertically downwards.

FIG. 4B is a schematic representation of a transverse section of a container formed by two thermoformed sheets, the container being provided, on the peripheral wall of one of the thermoformed sheets, with one access route that is oriented vertically upwards.

FIG. 4C schematically shows the structure of an access route.

FIG. 4D schematically shows the structure of an access route associated with an inner reinforcement.

FIGS. 5A-D show various structures that the ridges.

### DETAILED DESCRIPTION

A container 1 according to the invention includes two sheets 2 and 3, lower and upper respectively, which are secured to one another in the vicinity of their periphery.

According to one possible embodiment, sheets 2 and 3 may be welded. However, sheets 2 and 3 may also be secured by a different method, in particular by adhesive bonding. Container 1 thus defines an interior volume that is intended to receive cells and a culture medium.

Two sheets 2 and 3 may be permeable to gases, in particular to oxygen, and may be made of a transparent, biocompatible polymer material to which cells may adhere. As such, container 1 may allow very good development of cells, and its transparency offers the possibility of monitoring cell production using optical microscopy.

By way of example, the polymer material used to form sheets 2 and 3 may be made of polyester, in particular in APET or PETG form, polycarbonate or polystyrene. Moreover, the potential for cell adhesion of the polymers used for sheets 2 and 3 may easily be increased by various known surface treatments, in particular chemical grafting, or a treatment using activating gases. Preferably, a surface treatment of the plasma type (plasma/oxygen or plasma/air) may be carried out specifically on the surface intended for culturing. This may be done before container 1 is closed.

According to another embodiment of the invention, at least lower sheet 2 may be thermoformed. Container 1 may have, in transverse section, the overall shape of a rectangle with rounded comers. It may include a wall that forms bottom 4 of container 1, the wall being surrounded by side wall 5 which is extended laterally by peripheral wall 6 that forms a rim intended to be secured to upper sheet 3. The presence of rounded comers guarantees optimal recovery of the cell products after culturing.

Sheets 2 and 3 may have variable thicknesses and variable levels of gas permeability. For example, the thickness of one sheet may be between 100 and 500 μm. This small thickness makes it possible to obtain a satisfactory permeability to gases, in particular to oxygen. Additionally, thermoforming leads to a reduction in the thickness of the sheet. Therefore it is then possible to vary thermoforming so as to vary the gas permeability of container 1.

Moreover, the production of containers with various depths also makes it possible to vary the oxygenation of the medium. This is useful because certain cells, in particular haematopoietic cells, grow preferably in a medium with little oxygen. When growing such cells it is judicious to use a container of small depth that is filled exclusively with culture medium, so as to limit the exchange of gases. On the other hand, other types of cells, such as hepatocytes, consume a large amount of oxygen. To grow these cells it is recommended to use a deeper container that is partially filled with culture medium. This makes it possible to obtain an interface with a volume of air contained in the container, and consequently to promote the exchange of gases and in particular the supply of oxygen. In this context, the use of the thermoforming technique makes it possible to produce containers of various depths very easily, without it being necessary to create a mold for each type of container that is to be produced. A modification of the calibration of the mold is all that is required to modify the interior volume of the container.

According to another embodiment, the invention relates to a system including several elements, at least one of which may be a container as described above. The elements may be associated with one another so as to form a closed circuit. Such a system may in particular include elements that can sample, transfer, feed, concentrate, filter, inactivate or wash cell products. For example, these elements may include of flexible pouches for packaging media and reagents for cell culturing and flexible pouches for transferring and centrifuging the cell products. In this context, the concept of a closed system that incorporates at least one container according to the invention may make all of the handling operations that are carried out to produce cells by culturing much safer.

It may be judicious to limit the gas permeability of container 1 when the latter is integrated in a system capable of directing the supply of gases to cells. This permeability may be easily limited, in particular by using thicker sheets.

Directing of the fluids within the system defines a cell culture bioreactor. In this context, the system is able to continuously or sequentially supply the cells being cultured by circulating the media and reagents. The system may also be equipped with a set of regulation and control means. These means make it possible in particular to adjust the values of time, temperature, pH of the medium and gas content that are selected for a given application. A directable system or bioreactor is most particularly beneficial for carrying out long-term cell culturing. For example, it may be used for the production of mesenchymal cells extracted from bone marrow or for the production of haematopoietic cells in co-culture on adherent stromal cells.

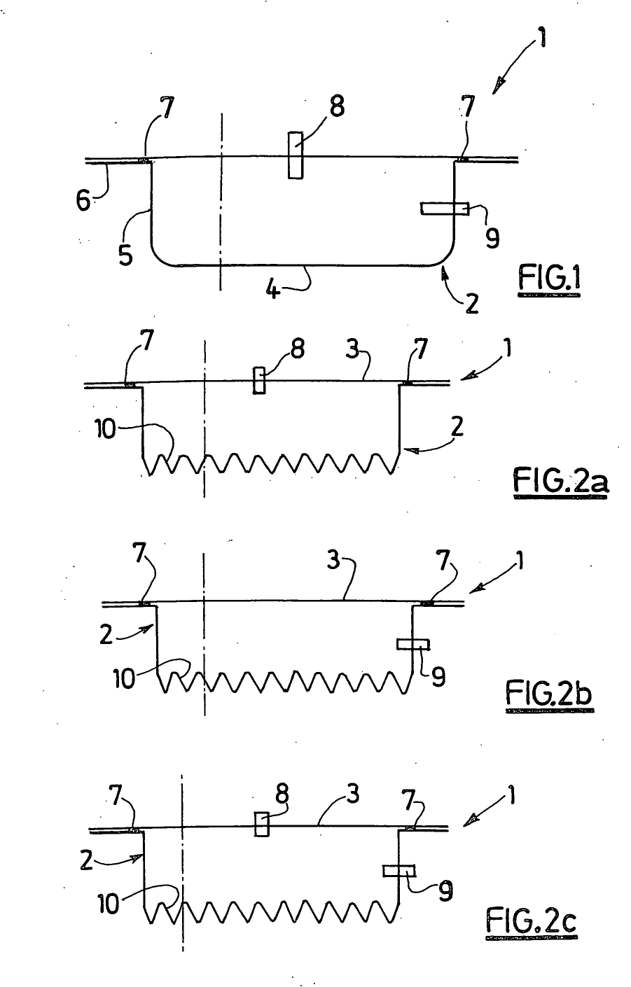

According to one embodiment of the invention, shown in FIGS. 1 and 2, upper sheet 3 is not thermoformed. Sheets 2 and 3 are secured to one another at welding zone 7 located, for example, on peripheral wall 6 of lower sheet 2, near side wall 5. Upper sheet 3 may be arranged so as to be substantially flat.

In FIG. 1, lower sheet 2 does not have ridges. Bottom 4, in particular, offers a homogeneous surface for the distribution and culturing of the cells because it is substantially flat and smooth.

Moreover, container 1 includes two orifices designed to allow the introduction and/or the recovery of cells by way of access routes cooperating with the orifices. First access route 8 communicates with the interior of container 1 through the upper wall of container 1. The upper wall may be formed by upper sheet 3. Second access route 9 communicates with the interior of container 1 through side wall 5 of thermoformed lower sheet 2.

In FIG. 2, lower sheet 2 has ridges 10 on its face that lies within container 1 essentially on bottom 4. Bottom 4 retains a flat and homogeneous overall shape. Ridges 10 make it possible to solve the problem of the large surfaces needed to culture certain cells in a closed system. Large surfaces are needed because the production of adherent human cells (mesenchymal, muscular, neural cells, etc.) is limited by the maximum density of the cells per unit surface. Once this limit is reached, cell proliferation ceases. The minimum number of cells required for a graft therefore necessitates a minimum cell culture surface, the latter usually having to be greater than one square meter. Thus it is beneficial to form ridges 10 on bottom 4 within container 1. Production of such ridges 10 on side walls 5 is possible, but may not be as useful because the cells will settle on bottom 4 due to gravity.

Thermoforming makes it possible to structure the cell culture surface and to considerably increase the available culture surface for a container without increasing external dimensions.

In one particular embodiment, sheets 2, 3, which may be thermoformed and structured with ridges, are provided with flat zones in order to facilitate the observation of the cells under a microscope and the insertion of access routes. Ridges 10 may take various forms, as will be described below with reference to FIG. 5.

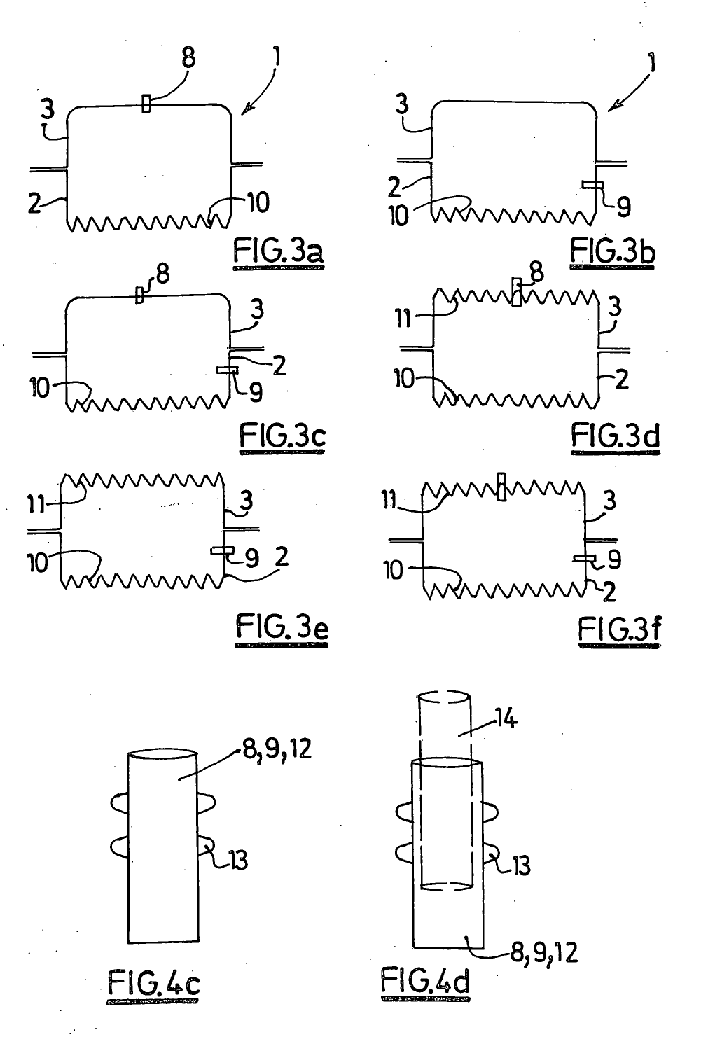

Container 1 as shown in FIG. 3 may be formed by thermoformed lower sheet 2 and thermoformed upper sheet 3. Upper sheet 3 may have a geometry that is the same as or substantially identical to that of lower sheet 2. Sheets 2 and 3 may be secured facing one another.

Lower sheet 2 may have ridges 10 on its face within container 1. Upper sheet 3 may also to have ridges 11 on its face within container 1 (FIGS. 3D-F) or, by contrast, it may have a surface that is substantially flat and smooth (FIGS. 3A-C). When two sheets 2 and 3 have ridges 10 and 11, container 1 may be placed either on lower sheet 2 or on upper sheet 3 in order to culture cells by adhesion. The culturing of adherent cells may then occur through contact with two sheets 2 and 3 at the same time, which doubles the already optimized capacity for cell production of container 1.

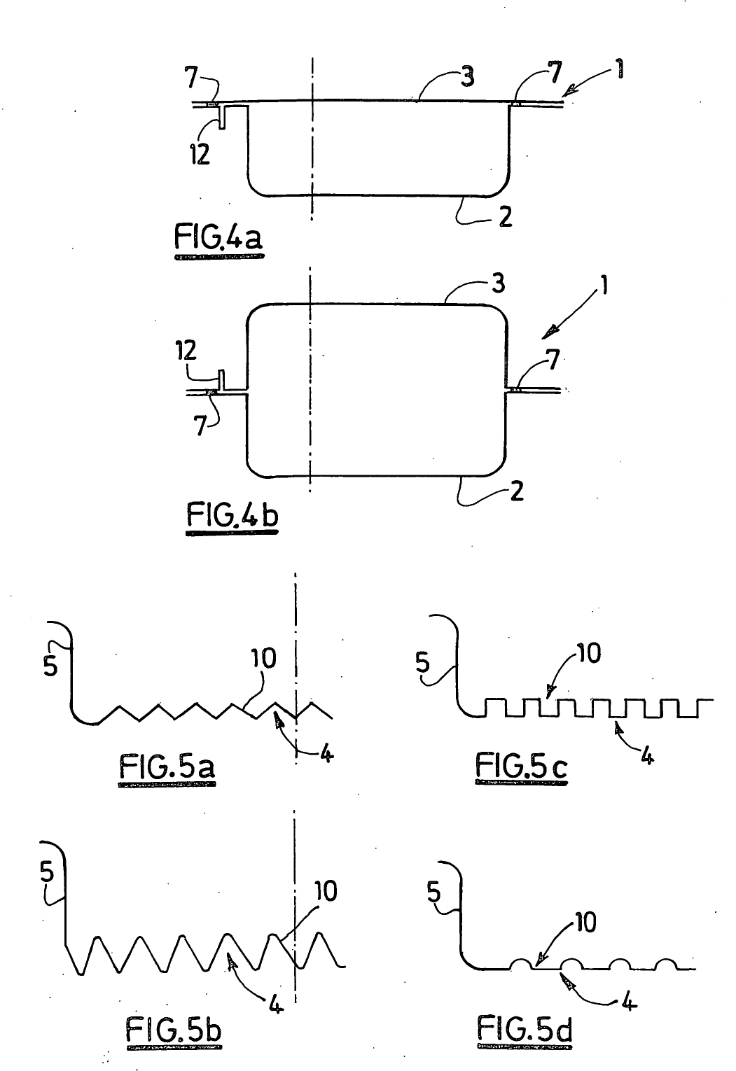

FIG. 4A shows container 1 formed by thermoformed lower sheet 2 that does not have ridges and by upper sheet 3, the sheets being secured to one another at welding zone 7. Upper sheet 3 may be arranged so as to be substantially flat. FIG. 4B is similar to FIG. 4A, although upper sheet 3 is thermoformed and does not have ridges.

Various embodiments may be used as regards the access routes. Container 1 may thus include one access route 8 that communicates with the interior of container 1 through the upper wall of container 1 that is formed by upper sheet 3 (FIGS. 2A, 3A and 3D). It may alternatively include one access route 9 that communicates with the interior of container 1 through side wall 5 of lower sheet 2 (FIGS. 2B, 3B and 3E). Container 1 may also contain both access routes 8 and 9 (FIGS. 1, 2C, 3C and 3F).

Container 1 may also have access route 12 associated with peripheral wall 6 of lower sheet 2 (FIG. 4A) or with the peripheral wall of upper sheet 3 (FIG. 4B).

Access routes 8 and 9 may be welded to sheets 2 and 3 but may also be secured to sheets 2 and 3 in particular by adhesive bonding. However, these peripheral access routes 8, 9 and 12 may also be produced by thermoforming.

First a protuberance may be created on the wall of sheet 2 or 3. Then, the protuberance may be perforated so as to create access routes 8, 9 or 12 and in particular allow a tube to be connected.]

According to one possible embodiment, shown in FIGS. 1, 2B, 2C, 3B, 3C, 3E and 3F, the protuberance may be created on the side wall defined by thermoforming of lower sheet 2. The use of thermoforming molds provided with removable parts makes it possible to extract the part thus formed.

According to another possible embodiment, shown in FIGS. 4A and 4B, the protuberance may be created on the sheet so as to be oriented perpendicular to the peripheral zone of the sheet. This orientation may facilitate easier production because the use of molds provided with removable parts is then no longer necessary.

The production of the container according to an embodiment of the invention and of its access routes 8, 9 or 12 makes it possible to circumvent the insertion of the access routes between the two sheets forming the container, as is the case in the manufacture of a flexible pouch. The zone where the two sheets are secured therefore remains entirely flat, and this constitutes a benefit for welding materials that are adhesive for cells, in particular non-complexed sheets including polyester, polycarbonate or polystyrene films.

Furthermore, the integration of access routes 8, 9 or 12 in at least one thermoformed sheet makes it possible to eliminate the risks of leakage which may exist at the zone where the routes are inserted between the sheets of the containers of the prior art.

The production of container 1 does not exclude the possibility of inserting the access routes, particularly in the form of tubes, between two sheets 2 and 3 (not shown). When low-flexibility polymer films are used, it may be necessary to preform therein the zones where these tubes will be located. This preforming may occur during thermoforming of sheets 2 and 3.

As shown in FIG. 4C, it is conceivable to add flutes 13 to access routes 8, 9 or 12 so as to improve the degree with which it is watertight with respect to a tube. Fluted end pieces may thus be provided. It is also conceivable to structure olive-shaped conical end pieces to achieve the same result.

As shown in FIG. 4D, it is also conceivable to add internal reinforcement 14 into access routes 8, 9 or 12, thus also making it possible to obtain a more watertight connection to a tube.

The sheets used may be of a small thickness so as to provide maximum permeability to gases, in particular to oxygen, and the thermoforming process further reduces this thickness. It may therefore be necessary to reinforce access routes 8, 9 and 12 so as to improve their solidity and also render them more watertight once they have been connected to a tube.

Reference is now made to FIG. 5, which shows various possible shapes of ridges 10 and 11.

Ridges 10 and 11 may in particular be in the form of corrugations, ripples, notches or burrs, which are shown respectively in FIGS. 5A, 5B, 5C and 5D. Ridges 10 and 11 may form repeating motifs or they may be irregular. Ridges 10 and 11 may extend over part or all of bottom 4 of container 1.

In order to obtain sufficient increases in the culture surface, ridges 10 and 11 are preferably not produced on a micrometric or nanometric scale but rather at least on a millimetric scale.

As mentioned above, the production of ridges 10 and 11 on the face of sheets 2 and 3 within container 1 by thermoforming makes it possible to increase the culture surface for adhering cells.

These ridges may also be a means of retaining non-adherent cells on sheet 2 and/or 3 as the medium is being circulated in the context of a cell culture with continuous perfusion of medium within container 1. In this respect, ridges having a notched shape, shown in FIG. 5C, the notches being arranged in the interior volume of container 1, are particularly suitable for culturing non-adherent cells with perfusion of the medium.

The technique of thermoforming has been developed for use in producing containers according to the invention. The containers according to the invention are innovative in that they provide a global response to all of the limiting criteria in the use of a flexible pouch for cell culturing. Furthermore, the technology for transforming plastics materials is particularly well suited to the production of a range of cell culture containers, the dimensional and structural characteristics of which may easily be adapted as a function of the cell types and their applications. Although they are particularly well suited to the preparation of cells for therapeutic purposes, containers container according to the invention may also he used for other biotechnological applications.

1. A cell culture container comprising:
a first sheet having a periphery;
a second sheet having an periphery and secured to the first sheet at their peripheries to form an interior volume operable to receive cells;
and at least one access route operable to allow the introduction and the recovery of the cells;
wherein at least one of the first and second sheets is gas-permeable;
wherein at least one of the first and second sheets includes at least one layer of a polymer material to which the cells may adhere; and
wherein at least one of the first and second sheets is thermoformed.

2. The container of claim 1, wherein both sheets include at least one layer of a polymer material to which the cells may adhere.

3. The container of claim 2, wherein both sheets consist essentially of a polymer material to which the cells may adhere.

4. The container of claim 1, wherein at least one sheet has in transverse section, the overall shape of a rectangle comprising:
a substantially flat bottom;
a side wall; and
a peripheral wall, the peripheral wall forming a rim.

5. The container of claim 4, wherein the at least one sheet forms an upper wall.

6. The container of claim 5, wherein the sheet forming the upper wall has a rectangular geometry.

7. The container of claim 1, further comprising at least one access route, wherein the access route communicates with the interior volume of the container through a wall of a sheet.

8. The container of claim 1, wherein at least one sheet comprises ridges arranged in the interior volume of container.

9. The container of claim 8, wherein the ridges comprise motifs that may be repeating or irregular and continuous or separate.

10. A system for cell culture comprising at least two elements connected in a closed circuit by a tube, wherein at least one of the elements comprises a container including:
a first sheet having a periphery;
a second sheet having an periphery and secured to the first sheet at their peripheries to form an interior volume operable to receive cells;
and at least one access route operable to allow the introduction and the recovery of the cells;
wherein at least one of the first and second sheets is gas-permeable;
wherein at least one of the first and second sheets includes at least one layer of a polymer material to which the cells may adhere;
wherein at least one of the first and second sheets is thermoformed.

11. The system of claim 11, further comprising the tube connected at a first end to an access route of the container and at a second end to another element of the system, so as to allow the cells and/or fluids to pass between the elements of the system.

12. The system of claim 11, wherein the system is operable for culturing of adherent cells or cells that are in suspension in the medium.

13. A method of producing a container comprising:
securing a first sheet having a periphery to a second sheet having an periphery at their peripheries to form an interior volume operable to receive cells; and
thermoforming at least one of the first and second sheets; and
providing at least one access route operable to allow the introduction and the recovery of the cells;
wherein at least one of the first and second sheets is gas-permeable; and
wherein at least one of the first and second sheets includes at least one layer of a polymer material to which the cells may adhere.

14. The method of claim 13, wherein securing comprises welding.
